(19) United States
(12) Patent Application Publication
(10) Pub. No.: US 2011/0189295 A1
(43) Pub. Date: Aug. 4, 2011

(54) TELMISARTAN TABLETS

(76) Inventors: Alena Prokopova, Praha (CZ); Eva Gryczova, Cernosice (CZ); Vaclav Tomasek, Praha (CZ)

(21) Appl. No.: 13/055,462

(22) PCT Filed: Jul. 28, 2009
(86) PCT No.: PCT/CZ2009/000096

§ 371 Date: Apr. 13, 2011

(30) Foreign Application Priority Data
Jul. 31, 2008 (CZ) PV-2008-469

(51) Int. Cl. A61K 31/4184; A61K 9/14

(52) U.S. Cl. 424/489; 514/394

(57) ABSTRACT

A composition containing the active substance telmisartan, which consists of granules of a telmisartan mixture, in which there is the active substance in the form of alkali salts, further contained is an organic or inorganic base selected from meglumine, sodium or potassium hydroxide, or a mixture of said bases, a binder, most preferably polyvinylpyrrolidone, sorbitol, and optionally other auxiliary substances; the composition further containing, outside the granules, particles of sorbitol, and optionally of other auxiliary substance, the size of 99% by\ weight of all particles of the telmisartan mixture being smaller than 1.0 mm and the size of 95% by weight of all particles of sorbitol contained in the composition inside as well as outside the granules of the telmisartan mixture being within the range of 0 to 0.250 mm.

### TECHNICAL FIELD

The invention relates to a solid pharmaceutical composition of telmisartan.

### BACKGROUND ART

According to WO 0027397, a spray-dried granulate of telmisartan with sorbitol is mixed with a lacidipine granulate and after addition of a lubricant the mixture is tabletted.

Document no. US 2005/0089575 A1 deals with the issue of compatibility of telmisartan and hydrochlorothiazide. It considers layered tablets as the most convenient solution. The first layer represents amorphous sodium salt of telmisartan and the other one hydrochlorothiazide (or a diuretic in general). These layered tablets make it possible both to solve poor solubility of telmisartan and the necessity to immediately release HCTZ.
According to the cited document the telmisartan layer is obtained by preparation of an aqueous solution of telmisartan together with a basic substance and a solubilizer. Then, this solution is spray-dried to provide spray-dried granulate. Finally, the granulate is mixed with fillers and a lubricant.
The hydrochlorothiazide layer is prepared by mixing and granulating HCTZ with components of the disintegrating tablet.
The premixes are compressed into the layered tablet by a known method.

According to WO 04028505 a surfactant, particularly a poloxamer, is added to the composition to further improve the telmisartan formulation. Again, either spray-drying of telmisartan alone is used or telmisartan is sprayed onto a solid carrier constituted of e.g. a sugar alcohol.

The known method of preparation of a pharmaceutical composition with telmisartan can be summarized as the stage of spray-drying of a solution of telmisartan and a base alone or with addition of a solid diluent and the stage of mixing with other ingredients. Whenever further active substances are used, another granulate with said substance is used, which is either mixed with the telmisartan granulate or is compressed into layered tablets.

Unfortunately, experience shows that when particular alternatives of this generally outlined method are applied in practice, the speed of releasing of the active substance varies in individual cases or even from batch to batch.

Therefore, the task was to find such a method of production of tablets containing telmisartan and possibly another active substance that will ensure reproducible properties of the product in a wide range of doses of the active substance.

### DISCLOSURE OF INVENTION

The invention provides a composition containing telmisartan as the active substance, which consists of granules of a telmisartan mixture, where there is the active substance in the form of alkali salts, an organic or inorganic base, selected from meglumine, sodium or potassium hydroxide, or a mixture of such bases, a binder, most suitably polyvinylpyrrolidone, sorbitol, and optionally other auxiliary substances; the composition further containing, outside said granules, particles of sorbitol, and optionally of other auxiliary substances, wherein the size of 99% by weight of all particles of the telmisartan mixture is smaller than 1.0 mm and the size of 95% by weight of all particles of sorbitol contained in the composition inside as well as outside the granules of the telmisartan mixture lies within the range of 0 to 0.250 mm.

In a preferable embodiment 70 to 80% by weight of the granules of the telmisartan mixture are in the size range of 0.1 to 0.8 mm and 60 to 70% by weight of sorbitol particles contained in the composition are in the size range of 0 to 0.125 mm.

Another factor influencing the speed of releasing of the active substance is the size of particles of this substance. It is obvious that the more uniform the distribution of said sizes, the more reproducible its release. In the case of the telmisartan composition according to the invention it has turned out that the most preferred are sizes of telmisartan particles in the range of 0.5 to 5 μm, which may form clusters, but not larger than 50 μm.

Furthermore, for an especially advantageous embodiment PVP (polyvinylpyrrolidone; Povidone) of type 25 is selected in the amount of 8.3% by weight, based on the whole composition.

This invention further provides a process of obtaining the above-described composition, consisting in dissolving telmisartan in a sodium or potassium hydroxide solution, adding the given amount of PVP and spraying the solution onto a fluidized layer of sorbitol.

### DETAILED DESCRIPTION OF THE INVENTION

Although the telmisartan composition is known, it has always been a problem to find such parameters of the composition which would provide reproducible results of release of the active substance (dissolution). Experiments have shown that even with the same constitution of the composition and with maintaining of the same procedure its characteristics, in particular the speed of release of the active substance, significantly change from batch to batch.

The speed of release of the active substance (dissolution) is one of the substantial factors influencing the concentration profile of the active substance in blood plasma. This factor is also related to biological availability of the pharmaceutical agent, i.e. the fraction of the total dose which is absorbed by the organism.

Therefore, ensuring reproducibility of release of the active substance is an absolutely necessary task of the development of a composition.

However, after ensuring reproducibility of dissolution it must be adjusted to such a level so that an effective concentration can be maintained in blood plasma throughout the period between two doses. It must also be ensured that this concentration will not exceed the limit that could have a negative impact on the patient in any case.

So another task consists in precisely establishing at what speed the substance should be released from the composition and how to exactly ensure such release.

Accordingly, the composition of the present invention is determined by parameters that lead to maintaining of reproducibility of dissolution and, in a preferred embodiment, by such parameters that will ensure specific values of dissolution or its time dependence in such a way as to achieve the desired final effect.

It has been found out that, in order to achieve the desired effect, it is necessary to produce such composition which would achieve the following values under the conditions described in Pharm. Eur, by the basket method at 100 rpm:

In an acidic environment of 0.01 M HCl the following limits have to be met:

| Time | Dissolved active substance |
|---|---|
| [min] | [%] |
| 15 | 25-55 |
| 30 | 55-85 |
| 45 | >85 |

and in the environment of a phosphate buffer at pH 6.8

| Time | Dissolved active substance |
|---|---|
| [min] | [%] |
| 15 | 45-75 |
| 30 | >80 |

For the dissolution speed of any substance, the size of its surface, given by the size of the active substance particle, is quite an obvious parameter. The smaller the particles are, the larger surface they provide and it can be justifiably assumed that they will dissolve faster. Therefore, it is quite obvious and is not explicitly mention in the description of the invention that all the parameters must be met at a constant size of particles of the active substance.

It has surprisingly turned out that the size of granules in which telmisartan is found is a substantial parameter for ensuring reproducibility of dissolution. Almost all granules must be smaller than 1 mm.

A second condition for reproducibility of dissolution is the size of free particles of sorbitol in the composition. They must almost all be smaller than 0.250 mm.

However, it has turned out that in order to improve further characteristics of the composition it is advantageous if the granules of the telmisartan mixture are distributed in the size of 0.1 to 0.8 mm to the extent of ¾ of the total weight. Similarly, as regards free sorbitol it is preferable if its particles are smaller than 0.125 mm to the extent of ⅔ of the used substance weight.

The weight fractions of individual particle sized are determined by grain size analysis, the result of which is described in more detail in the examples mentioned below.

In a preferable embodiment the active substance is located in the intragranular space. In particular, the granules are bound by a solution, which contains the PVP binder, an alkali salt of telmisartan and an alkali hydroxide, and optionally meglumine. From the point of view of further characteristics of the product the composition wherein meglumine is outside the granules of the telmisartan mixture has proved to be the more convenient.

The method for preparing the composition described below, i.e. spraying of the active substance on a solid carrier, is known as a method that can provide a relatively narrow distribution of particles, which can be set by means of parameters of the given apparatus. However, in the case of the given telmisartan mixture it turns out that the particles tend to form clusters. For the desired dissolution, particles with the size of 0.5 to 5 μm with smaller clusters than 50 μm appear to be convenient. A usual setting of particles is about 2 μm and clusters 30-50 μm. It is obvious that the composition of the invention may also exhibit the desired dissolution with other ratios of the particle/cluster sizes.

The sizes of particles of the active substance in the granulate can be determined by means of the Raman or IR spectroscopy methods, in particular the method known as mapping.

In the course of the development of the composition in accordance with the invention it has further turned out that the PVP binder used importantly participates in the final dissolution of the active substance. For successful setting of parameters various types of PVP can be used. But the amount of each type must be determined separately. Polyvinylpyrrolidone of types 25, 30 or 90, i.e. of the molecular weights 30,000, 50,000 and 1,000,000, resp., can be taken into consideration. In the case of using PVP of type 25, 30 or 90 the optimum values are in the range of 2 to 10% by weight. PVP of type 25 appears to be convenient in the amount of 6 to 10%, more preferably in the amount of 8.3±0.5% b\ weight.

### Production Procedure:

Telmisartan is dissolved in water, preferably in a water/ethanol mixture, in the presence of basic substances such as NaOH, KOH, meglumine, and the like, preferably only NaOH, and a binder, which is PVP, which serves not only as a binder, but also as a stabilizer for the solution. This solution is used for fluid granulating the sorbitol. The dried and sieved granulate is mixed with a part of the soluble filler, sorbitol and other excipients, such as meglumine or magnesium stearate.

Particular parameters of the fluid process, such as the temperatures of the fluidized feed medium (e.g. air), the speed of spraying of the telmisartan solution onto the fluidized layer or the size of sprayed particles, have to be set with regard to the particular fluid granulation apparatus. Examples of particularly selected granulation parameters are mentioned below; however, they cannot be used for any apparatus; they only serve to illustrate the invention. An important parameter which must be met in any such apparatus is the size of sorbitol particles before the start of granulation. Almost all these particles must be smaller than 250 μm and it is preferable if about % of them are smaller than 0.125 mm.

### EXAMPLES

In all the examples mentioned below sorbitol in particles out of which 98% by weight were smaller than 0.250 mm and 67% were smaller than 0.1 mm was used for preparation of the composition.

Composition A: Telmisartan 80 mg tbl.

| Composition | mg/tbl. |
|---|---|
| Telmisartan | 80.0 |
| Sodium hydroxide | 6.8 |
| Povidone | 40.0 |
| Sorbitol | 324.4 |
| Meglumine | 24.0 |
| Magnesium stearate | 4.8 |
| Total weight of the core | 480.0 |

The total charge was 10 kg.

Telmisartan was dissolved in a solution of NaOH and povidone. Sorbitol was fluid granulated with the resulting solution. The granulation proceeded in the WSG 15 apparatus with the average spraying speed of 10 g/min.kg of sorbitol at the input air temperature in the range of 47 to 55° C. and the product temperature in the range of 30 to 40° C. The resulting granulate was fluid dried. After drying the granulate was sieved and subsequently mixed with the remaining quantity of sorbitol, meglumine and magnesium stearate. Cores with the weight of 480 mg were compressed from the resulting tabletting matter. The produced cores released 39% of the active substance after 15 minutes, 72% of the effective substance after 30 minutes and 95% after 45 minutes in the dissolution apparatus according to Ph.Eur in 0.01 M of HCl.

The granulate produced by the above mentioned method provided the following sieve analysis:

| | | | | | | |
|---|---|---|---|---|---|---|
| Size [mm] | <0.1 | 0.1-0.25 | 0.25-0.5 | 0.5-0.8 | 0.8-1.0 | >1.0 |
| Fraction [% by weight] | 8.3 | 10.4 | 32.4 | 39.7 | 9.1 | 0.2 |

Composition B: Telmisartan 80 mg tbl.

| Composition | mg/tbl. |
|---|---|
| Telmisartan | 80.0 |
| Sodium hydroxide | 6.8 |
| Povidone | 40.0 |
| Sorbitol | 348.4 |
| Meglumine | 0 |
| Magnesium stearate | 4.8 |
| Total weight of the core | 480.0 |

The total charge was 1 kg.

Telmisartan was dissolved in a solution of NaOH and povidone. Sorbitol was fluid granulated with the resulting solution. The granulation proceeded in the Aeromatic MP1 machine with the average spraying speed of 21 g/min.kg of sorbitol at the input air temperature in the range of 47 to 55° C. and the product temperature in the range of 33 to 34° C. The resulting granulate was fluid dried. After drying the granulate was sieved and subsequently mixed with the remaining quantity of sorbitol and magnesium stearate. Cores with the weight of 480 mg were compressed from the resulting tabletting matter. The produced cores released 26% of the active substance after 15 minutes, 44% of the active substance after 30 minutes and 57% after 45 minutes in the dissolution apparatus according to Ph.Eur in 0.01 M of HCl.

Granulate Size after Sieving:

| | | | | | | |
|---|---|---|---|---|---|---|
| Size [mm] | <0.1 | 0.1-0.25 | 0.25-0.5 | 0.5-0.8 | 0.8-1.0 | >1.0 |
| Fraction [% by weight] | 10.2 | 34.8 | 47.6 | 7.2 | 0.2 | 0 |

Composition C: Telmisartan 80 mg tbl.

| Composition | mg/tbl. |
|---|---|
| Telmisartan | 80.0 |
| Sodium hydroxide | 6.8 |
| Povidone | 40.0 |
| Sorbitol | 343.6 |
| Meglumine | 4.8 |
| Magnesium stearate | 4.8 |
| Total weight of the core | 480.0 |

The total charge was 1 kg.

Telmisartan was dissolved in a solution of NaOH and povidone. Sorbitol was fluid granulated with the resulting solution. The granulation proceeded in the Aeromatic MP1 machine with the average spraying speed of 18 g/min. kg of sorbitol at the input air temperature in the range of 47 to 55° C. and the product temperature in the range of 35 to 36° C. The resulting granulate was fluid dried. After drying the granulate was sieved and subsequently mixed with the remaining quantity of sorbitol, meglumine and magnesium stearate. Cores with the weight of 480 mg were compressed from the resulting tabletting matter. The produced cores released 30% of the active substance after 15 minutes, 52% of the active substance after 30 minutes and 69% after 45 minutes in the dissolution apparatus according to Ph.Eur in 0.01 M of HCl.

Granulate Size after Sieving:

| | | | | | | |
|---|---|---|---|---|---|---|
| Size [mm] | <0.1 | 0.1-0.25 | 0.25-0.5 | 0.5-0.8 | 0.8-1.0 | >1.0 |
| Fraction [% by weight] | 13.6 | 52.4 | 32.6 | 1.2 | 0.2 | 0 |

1. A solid pharmaceutical composition, comprising: granules of a telmisartan mixture, wherein the active substance in the form of alkali salts; an organic or inorganic base selected from the group consisting of meglumine, sodium hydroxide and potassium hydroxide and their mixtures, a binder, which is polyvinylpyrrolidone or sorbitol; wherein the composition further comprises, externally to the granules, particles of sorbitol, wherein the size of 99% by weight of all granules of the telmisartan mixture is smaller than 1.0 mm and the size of 95% by weight of all the particles of sorbitol, both bound to the granules of the telmisartan mixture and externally to these granules, is smaller than 0.250 mm.

2. The pharmaceutical composition according to claim 1, wherein 70 to 80% by weight of granules of the telmisartan mixture is in the range of 0.1 to 0.8 mm and 60 to 70% by weight of the sorbitol particles is smaller than 0.125 mm.

3. The pharmaceutical compositions according to claim 1, wherein the telmisartan mixture consists of sorbitol particles, which are bound together to form a granulate by the effect of a solution of the telmisartan salt and the binder and a base consisting of sodium or potassium hydroxide, in a mixture with meglumine.

4. The pharmaceutical composition according to claim 1, wherein the binder is PVP of type 25, 30 or 90 in an amount of 2 to 10% by weight.

5. The pharmaceutical composition according to claim 4, wherein the binder is PVP of type 25 in an amount of 6 to 10 weight %.

6. The pharmaceutical composition according to claim 5, wherein the amount of PVP of type 25 is selected in the range of 8.3±0.5% by weight.

7. The pharmaceutical composition according to claim 3, comprising granules of the telmisartan mixture containing the telmisartan salt at a particle size of 0.5 to 5 μm, wherein the size of the clusters is less than 50 μm.

8. The pharmaceutical composition according to claim 3, wherein the telmisartan mixture comprises sodium or potassium hydroxide inter-granules and meglumine externally to the granules.

9. A process for the manufacture of the composition according to claim 1, comprising the steps of: spraying a solution comprising telmisartan in water, or water and ethanol; sodium or potassium hydroxide; a binder, and meglumine on a fluidized layer of sorbitol, wherein the dried granulate is mixed with another portion of sorbitol, meglumine, and other auxiliary substances.

10. The process according to claim 9, wherein meglumine is not contained in the sprayed telmisartan solution, but is admixed into the final granulate of the telmisartan mixture.

11. The process according to claim 9, wherein a lubricant, selected from salts of stearic acid, talc or colloidal silica, is added to the granulate.

12. The process according to claim 9, wherein 0.5 to 3% by weight of sodium or potassium hydroxide and 10 to 20% by weight of telmisartan are sprayed and 1 to 10% by weight of meglumine and 0.5 to 3% by weight of magnesium stearate, based on the final weight of the composition, are further sprayed onto or added to the prepared granulate.

13. The process according to claim 12, wherein 1 to 2% by weight of sodium or potassium hydroxide, 4 to 8% by weight of meglumine, 10 to 20% by weight of telmisartan and 0.8 to 2.5% by weight of magnesium stearate, based on the final weight of the composition, are further sprayed onto or added to the prepared granulate.
